(19) United States
(12) Patent Application Publication
(10) Pub. No.: US 2025/0082765 A1
(43) Pub. Date: Mar. 13, 2025

(54) MOLECULES AS ANTICANCER AGENTS FOR TARGETED TREATMENT OF BREAST AND PROSTATE CANCER

(71) Applicant: Dr. Boggarapu Prakash RAO, Bangalore (IN)

(72) Inventors: Dr. Boggarapu Prakash RAO, Bangalore (IN); Buvanagiri SUDHA, Bangalore (IN); Sarasija SURESH, Bangalore (IN); Kalavathy Doddabandara JAVAREGOWDA, Bangalore (IN); Gutta Prabhakar RAO, Hyderabad (IN); Naga Venkata Lakshmi Sirisha MULUKURI, Bangalore (IN); Chandanam SREEDHAR, Bangalore (IN); Shashidharreddy DODDA, Hyderabad (IN)

(21) Appl. No.: 18/291,607

(22) PCT Filed: Jul. 26, 2022
(86) PCT No.: PCT/IB2022/056868

§ 371 Date: Jan. 24, 2024

(30) Foreign Application Priority Data
Jul. 26, 2021 (IN) 202141019126

(51) Int. Cl. A61K 47/54

(52) U.S. Cl. CPC A61K 47/554

(57) ABSTRACT

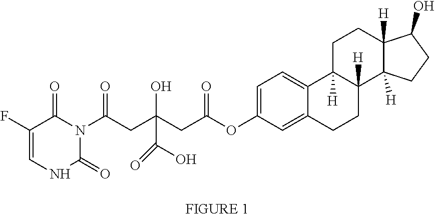

The invention discloses a molecule for targeting the specific receptors in mitigating breast cancer and prostate cancer. The magic bullet is derived as a small molecule using the molecular modelling technique. The molecule of formula (I) comprises the acid as first chemical moiety, hormone derivative as second chemical moiety and the anticancer agent as third chemical moiety. The acid is citric acid or tartaric acid. The hormone for the molecule of formula (I) is estradiol, genistein, apigenin or dihydroxy testosterone. The drug used is selected from 5-flurouracil, capecitabine, gemcitabine, 6-mercaptopurine or calibridin for treatment of breast cancer and 5-flurouracil for treatment of prostate cancer. The molecule of formula (I) is chemically stable, cost effective, exhibits improved efficacy due to specific target, extended duration of therapeutic activity without inducing any immune reaction.

### PRIORITY CLAIM

This application claims priority from the provisional application numbered 202141019126 filed with Indian Patent Office, Chennai on Jul. 26, 2021 entitled “Molecules as anticancer agents for targeted treatment of breast and prostate cancer”, the entirety of which is expressly incorporated herein by reference

### DESCRIPTION OF THE INVENTION

### Technical Field of the Invention

The present invention relates to a unique molecule with a combination of compounds for targeted therapy of cancer. More particularly, the invention relates to a derived small molecule drug as a magic bullet using a hormone related ligand for specific targeting in the treatment of breast cancer and prostate cancer.

### BACKGROUND OF THE INVENTION

Cancer refers to a condition involving abnormal cell growth with the potential to invade or spread to other parts of the body. Some types of cancer cause rapid cell growth, while others cause cells to grow and divide at a slower rate. Certain forms of cancer result in visible growth called tumors, while others, such as leukemia, do not. The possible signs and symptoms of cancer include a lump, abnormal bleeding, prolonged cough, unexplained weight loss, and a change in bowel movements.

There are different types of cancers based on the organ or tissue affected. Breast cancer is cancer that forms in the cells of the breasts. After skin cancer, breast cancer is the most common cancer diagnosed in women. Breast cancer can occur in both men and women, but more common in women. These cells divide more rapidly than healthy cells do and continue to accumulate forming a lump or mass. Cells may metastasize through breast to lymph nodes or to other parts of the body.

Genetic factors also contribute to the development of the cancer. Genes also influence the cells' production of proteins, and proteins carry many of the instructions for cellular growth and division. Some genes change proteins that would usually repair damaged cells, leading to cancer. Some genetic changes occur after birth, and factors such as smoking, and sun exposure increase the risk. Other changes that can result in cancer take place in the chemical signals that determine how the body deploys, or “expresses” specific genes.

Breast cancer most often begins with cells in the milk-producing ducts (invasive ductal carcinoma). Breast cancer may also begin in the glandular tissue called lobules (invasive lobular carcinoma) or in other cells or tissue within the breast.

Prostate cancer is cancer that occurs in the prostate, which is a small walnut-shaped gland in males that produces the seminal fluid that nourishes and transports sperm. Prostate cancer is one of the most common types of cancer. Many prostate cancers grow slowly and are confined to the prostate gland, where they may not cause serious harm. However, while some types of prostate cancer grow slowly and may need minimal or even no treatment, other types are aggressive and spread rapidly.

There are different modes of treatment available for cancers including chemotherapy, radiotherapy etc. These are based on the severity of the diseases and also on case-to-case basis. However, the long-term exposure or the high dose administration are associated with damage of the normal cells. Hence, targeted therapy has been an alternative approach for treatment of cancers.

Targeted therapy refers to the use of drugs or other substances or molecules to identify and attack specific types of cancer cells with less harm to normal cells. Some targeted therapies block the action of certain enzymes, proteins, or other molecules involved in the growth and spread of cancer cells. Targeted therapy affects the tissue environment that helps a cancer grow and survive or it target the cells related to cancer growth such as blood vessel cells.

A potential target for the therapy would be a protein that is present in cancer cells but not the healthy cells, which is caused by a mutation. Once the mutation is identified, a specific treatment is developed that targets the specific mutation.

Targeted therapy is achieved through various mechanisms such as by blocking the signals that communicate with cancer cells to grow and divide, prevent the cells from living longer duration than normal life span and finally destroys the cancer cells.

There are different types of targeted therapies such as use of monoclonal bodies, nanoparticles etc., that are used as targeted drug delivery systems or passive targeting by polyethylene glycol. However, the difficulties in conjugation of monoclonal antibodies to nanoparticles hinders their use in targeted therapy. In addition, the monoclonal bodies are not cost-effective and also do not cross cell membrane due to high molecular weight. Monoclonal antibodies receptor-targeting peptides, and some intracellular sorting signals are more specific regarding recognition of molecular signatures of cells, yet these elements often do not provide the ability to cross cellular membranes, posing obstacles to enter the cell cytosol, e.g., directly from the extracellular milieu or from endocytic compartments after endocytosis.

The Patent Application No. EA030958B1 entitled “Acc inhibitors and uses thereof” discloses the compounds and their pharmaceutically acceptable compositions suitable for the treatment of various diseases, disorders or conditions. The compounds proposed in accordance with the present invention may also be suitable for studying ACC enzymes in biological and pathological phenomena, studying the intracellular pathways of signal transduction occurring in lipogenic tissues and comparative evaluation of new inhibitors of ACC or other regulators of fatty acid levels in vitro or in vivo.

The Patent Application No. AU2016362239B2 entitled “Salts of conjugates for cancer therapy” discloses pharmaceutically acceptable salts of conjugates comprising a chemotherapeutic drug and an amino acid or a derivative thereof, which are readily taken up by a target cell and reduce side effects induced by the chemotherapeutic drug. In particular, the present invention relates to pharmaceutically acceptable salts of conjugates comprising cytidine analog drugs and aspartic or glutamic acid and analogs thereof, pharmaceutical compositions comprising these conjugates and use thereof for the treatment of cancer or a pre-cancer condition or disorder.

The Patent Application No. JP2019502663A entitled “(R)-1-(4-(6-(2-(4-(3,3-difluorocyclobutoxy)-6-methylpyridin-2-yl) acetamido)pyridazin-3-yl)-2-fluorobutyl)-N-methyl-1H-1,2,3-triazole-4-carboxamide salt and polymorphs” discloses compound (R)-1-(4-(6-(2-(4-(3,3-difluorocyclobutoxy)-6-methylpyridin-2-yl) acetamido)pyridazine-showing improved exposure after oral dosing 3-yl)-2-fluorobutyl)-N-methyl-1H-1,2,3-triazole-4-carboxamide and its salt forms and polymorphs along with methods of inhibiting GLS1 activity in a human or animals.

The Patent Application No. AU2019246728A1 entitled “Dipeptide piperidine derivatives” relates to pharmaceutical compositions, to methods of preparing such compositions, and to methods for using such compositions for treating or preventing a disease or condition associated with arginase activity.

The existing approaches of the targeted therapy are associated with lack of stability, high cost, difficulty in crossing the cell membrane due to high molecular weight, quality assurance and specific targeting. Hence, there is a need for a small molecule with a combination of different substances to specifically target the cells for treatment of breast cancer and prostate cancer.

### SUMMARY OF THE INVENTION

The present invention overcomes the drawbacks of existing prior art. The present invention relates to a molecule for the targeted therapy of breast cancer and prostate cancer. The molecule is of low molecular weight and does not induce any immune response.

The present invention discloses a molecule comprising an ester of acid, hormone derivative and an anticancer agent in combination. The acid is selected from citric acid and tartaric acid. The hormone derivate estradiol is incorporated for treating breast cancer and the hormone derivative dihydrotestosterone is incorporated for treating prostate cancer. The present invention discloses multiple combinations of the molecule of formula (I) comprising acid, hormone derivative and the anticancer agent.

The present invention further discloses biodistribution analysis of anticancer agent capecitabine in the plasma of mice models at various time intervals. The mice were administered with 229.91 mg of molecule of formula (I) comprising citric acid-capecitabine-estradiol for breast cancer and 227 mg of molecule of formula (I) comprising citric acid-capecitabine-dihydrotestosterone for prostate cancer.

The biodistribution analysis indicates the identification and estimation of capecitabine at the target organ. The biodistribution analysis is performed for target organs breast and prostate gland along with blood, liver, lungs and spleen. The average biodistribution indicates the presence of capecitabine at the target organs breast and prostate gland at various time intervals. The biodistribution of capecitabine was found to be negligible in liver, lungs and spleen at various time intervals.

The present invention discloses a molecule for achieving targeted therapy of breast and prostate cancer. The molecule comprising an acid, hormone derivative and an anticancer agent exhibits synergistic effect in the targeted therapy of breast and prostate cancer. The molecule is of low molecular weight, hence easily permeable through the plasma membrane, highly stable and does not induce any immune response. The molecule further exhibits extended therapeutic activity in mouse model.

### BRIEF DESCRIPTION OF DRAWINGS

FIG. 1 illustrates the structure of the molecule of formula (I) comprising 5-fluorouracil-citric acid-estradiol according to an embodiment of the invention.

FIG. 2 illustrates the structure of the molecule of formula (I) comprising capacitabine-citric acid-estradiol according to an embodiment of the invention.

FIG. 3 illustrates the structure of the molecule of formula (I) comprising cladibrine-citric acid-estradiol according to an embodiment of the invention.

FIG. 4 illustrates the structure of the molecule of formula (I) comprising ethionine-citric acid-estradiol according to an embodiment of the invention.

FIG. 5 illustrates the structure of the molecule of formula (I) comprising mercaptopurine-citric acid-estradiol according to an embodiment of the invention.

FIG. 6 illustrates the structure of the molecule of formula (I) comprising capacitabine-citric acid-dihydrotestosterone according to an embodiment of the invention.

FIG. 7 illustrates the structure of the molecule of formula (I) comprising mercaptopurine-citric acid-dihydrotestosterone according to an embodiment of the invention.

FIG. 8 illustrates the structure of the molecule of formula (I) comprising ethionine-citric acid-dihydrotestosterone according to an embodiment of the invention.

FIG. 9 illustrates the structure of the molecule of formula (I) comprising cladibrine-citric acid-dihydrotestosterone according to an embodiment of the invention.

FIG. 10 illustrates the structure of the molecule of formula (I) comprising 5-fluorouracil-citric acid-dihydrotestosterone according to an embodiment of the invention.

FIG. 11 tabulates the biodistribution of capecitabine in breast tissue of mouse.

FIG. 12 tabulates biodistribution of capecitabine in prostate gland of mouse.

FIG. 13 tabulates biodistribution of capecitabine in several organs of mouse.

### DETAILED DESCRIPTION OF THE INVENTION

In order to more clearly and concisely describe and point out the subject matter of the claimed invention, the following definitions are provided for specific terms, which are used in the following written description.

The term “Drug Target” refers to a molecule in the body, usually a protein, that is intrinsically associated with a particular disease process and that could be addressed by a drug to produce a desired therapeutic effect.

The term “Targeted Therapy” refers to cancer treatment that uses drugs to target specific genes and proteins that are involved in the growth and survival of cancer cells.

The term “Small Molecule” refers to any organic compound that affects a biologic process with a relatively low molecular weight i.e., below 900 Daltons.

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

The present invention overcomes the drawbacks of the existing technologies by providing a small molecule comprising a drug for targeted therapy for breast cancer and prostate cancer.

Accordingly, to an embodiment of the invention, a small molecule is disclosed as a magic bullet for targeting the specific receptors in mitigate the breast cancer and the prostate cancer. The magic bullet is derived as a small molecule using the molecular modeling technique.

The small molecule of the present invention is designed to comprise an acid, a hormone and an anticancer agent. The molecule of formula (I) is derived by using various combinations of these substances by altering the anticancer agent based on the type of the cancer.

The small molecule derived from various combinations of an acid comprising citric acid and tartaric acid, a hormone derivative comprising estradiol for breast cancer and dihydrotestosterone for prostate cancer and an anticancer agent comprising 5-fluorouracil, cladibrine, ethionine, mercaptapurine, capecitabine and gemcitabine.

The present invention comprises molecule of formula (I) or its pharmaceutically accepted salts comprising first chemical moiety, second chemical moiety and the third chemical moiety that are covalently linked to each other. The present invention discloses various combinations of molecules of formula (I).

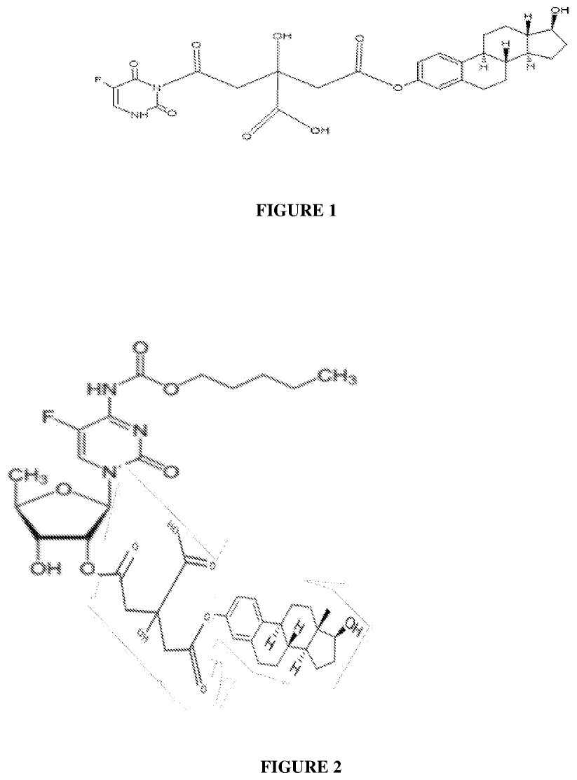

FIG. 1 illustrates the structure of the molecule of formula (I) comprising 5-fluorouracil-citric acid-estradiol according to an embodiment of the invention. The molecule of formula comprises citric acid as first chemical moiety, hormone derivative estradiol as second chemical moiety and the anticancer agent 5-fluorouracil as third chemical moiety. 5-fluorouracil-citric acid-estradiol is synthesized by esterification method using dimethylformamide (DMF) as a solvent. The esterification reaction is achieved by blocking the amine group using 1-Ethyl-3-(3-dimethylaminopropyl) carbodiimide (EDCI). In addition, EDCI functions as a cross-linking agent. The chemical moieties are covalently linked to one another. The molecular weight of 5-fluorouracil-citric acid-estradiol is 594.6.

FIG. 2 illustrates the structure of the molecule of formula (I) comprising capacitabine-citric acid-estradiol according to an embodiment of the invention. The molecule comprises citric acid as first chemical moiety, hormone derivative estradiol as second chemical moiety and the anticancer agent capacitabine as third chemical moiety. Capacitabine-citric acid-estradiol is synthesized by esterification method using DMF as a solvent. The esterification reaction is achieved by blocking the amine group using EDCI.

In addition, EDCI functions as a cross-linking agent. The chemical moieties are covalently linked to one another. The molecular weight of capacitabine-citric acid-estradiol is 781.84 g/mol.

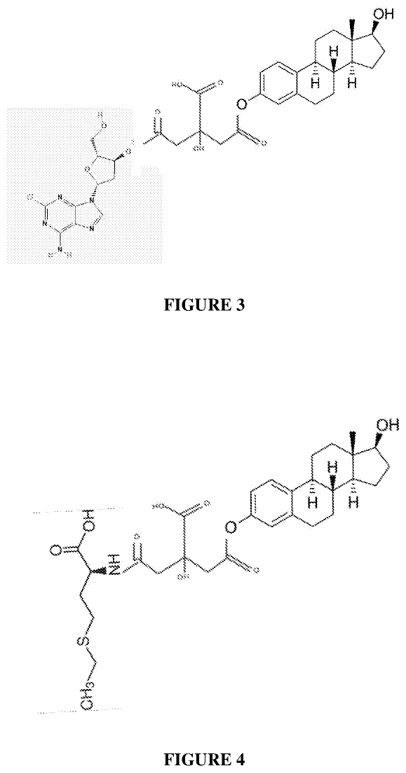

FIG. 3 illustrates the structure of the molecule of formula (I) comprising cladibrine-citric acid-estradiol according to another embodiment of the invention. The molecule comprises citric acid as first chemical moiety, hormone derivative estradiol as second chemical moiety and the anticancer agent cladibrine as third chemical moiety. Cladibrine-citric acid-estradiol is synthesized by esterification method using DMF as a solvent. The esterification reaction is achieved by blocking the amine group using EDCI. In addition, EDCI functions as a cross-linking agent. The chemical moieties are covalently linked to one another. The molecular weight of cladibrine-citric acid-estradiol is 750.21 g/mol.

FIG. 4 illustrates the structure of the molecule of formula (I) comprising ethionine-citric acid-estradiol according to another embodiment of the invention. The molecule comprises citric acid as first chemical moiety, hormone derivative estradiol as second chemical moiety and the anticancer agent ethionine as third chemical moiety. Ethionine-citric acid-estradiol is synthesized by esterification method using DMF as a solvent. The esterification reaction is achieved by blocking the amine group using EDCI. In addition, EDCI functions as a cross-linking agent. The chemical moieties are covalently linked to one another. The molecular weight of ethionine-citric acid-estradiol is 627.6 g/mol.

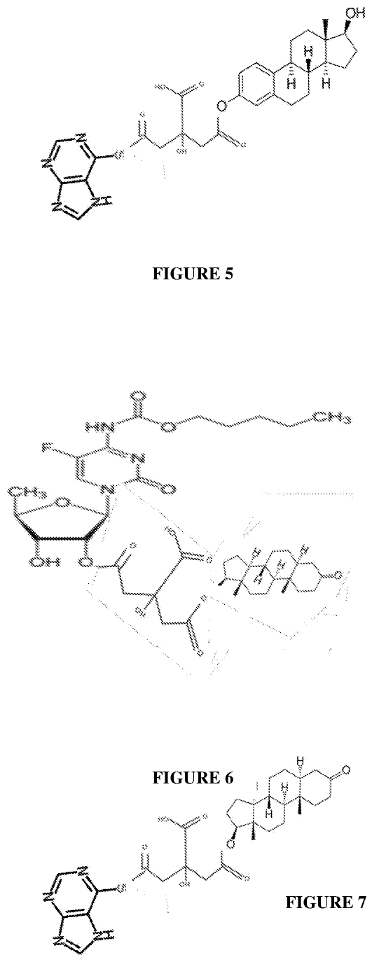

FIG. 5 illustrates the structure of the molecule of formula (I) comprising mercaptopurine-citric acid-estradiol according to another embodiment of the invention. The molecule comprises citric acid as first chemical moiety, hormone derivative estradiol as second chemical moiety and the anticancer agent mercaptopurine as third chemical moiety. Mercaptopurine-citric acid-estradiol is synthesized by esterification method using DMF as a solvent. The esterification reaction is achieved by blocking the amine group using EDCI. In addition, EDCI functions as a cross-linking agent. The chemical moieties are covalently linked to one another. The molecular weight of mercaptopurine-citric acid-estradiol is 594.6 g/mol.

FIG. 6 illustrates the structure of the molecule of formula (I) comprising capecitabine-citric acid-dihydrotestosterone according to another embodiment of the invention. The molecule comprises citric acid as first chemical moiety, hormone derivative dihydrotestosterone as second chemical moiety and the anticancer agent capecitabine as third chemical moiety. Capecitabine-citric acid-dihydrotestosterone is synthesized by esterification method using DMF as a solvent. The esterification reaction is achieved by blocking the amine group using EDCI. In addition, EDCI functions as a cross-linking agent. The chemical moieties are covalently linked to one another. The molecular weight of capecitabine-citric acid-dihydrotestosterone is 841.92 g/mol.

FIG. 7 illustrates the structure of the molecule of formula (I) comprising mercaptopurine-citric acid-dihydrotestosterone according to another embodiment of the invention. The molecule comprises citric acid as first chemical moiety, hormone derivative dihydrotestosterone as second chemical moiety and the anticancer agent mercaptopurine as third chemical moiety. Mercaptopurine-citric acid-dihydrotestosterone is synthesized by esterification method using DMF as a solvent. The esterification reaction is achieved by blocking the amine group using EDCI. In addition, EDCI functions as a cross-linking agent. The chemical moieties are covalently linked to one another. The molecular weight of mercaptopurine-citric acid-dihydrotestosterone is 634.74 g/mol.

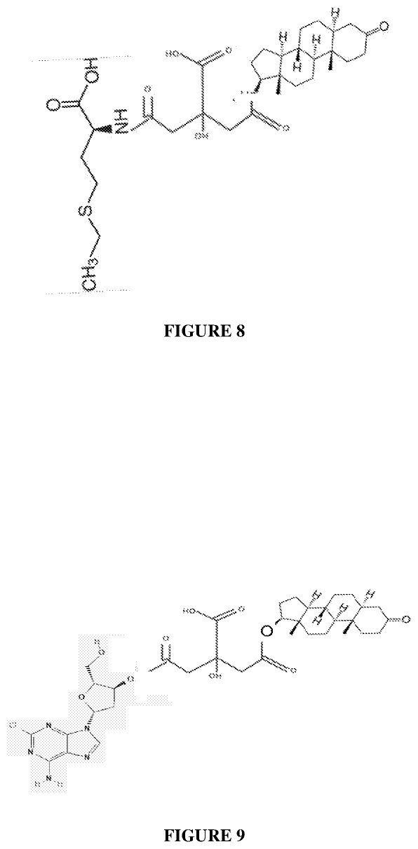

FIG. 8 illustrates the structure of the molecule of formula (I) comprising ethionine-citric acid-dihydrotestosterone according to another embodiment of the invention. The molecule comprises citric acid as first chemical moiety, hormone derivative dihydrotestosterone as second chemical moiety and the anticancer agent ethionine as third chemical moiety. Ethionine-citric acid-dihydrotestosterone is synthesized by esterification method using DMF as a solvent. The esterification reaction is achieved by blocking the amine group using EDCI. In addition, EDCI functions as a cross-linking agent. The chemical moieties are covalently linked to one another. The molecular weight of ethionine-citric acid-dihydrotestosterone is 645.8 g/mol.

FIG. 9 illustrates the structure of the molecule of formula (I) comprising cladibrine-citric acid-dihydrotestosterone according to another embodiment of the invention. The molecule comprises citric acid as first chemical moiety, hormone derivative dihydrotestosterone as second chemical moiety and the anticancer agent cladibrine as third chemical moiety. Cladibrine-citric acid-dihydrotestosterone is synthesized by esterification method using DMF as a solvent. The esterification reaction is achieved by blocking the amine group using EDCI. In addition, EDCI functions as a cross-linking agent. The chemical moieties are covalently linked to one another. The molecular weight of cladibrine-citric acid-dihydrotestosterone is 768.25 g/mol.

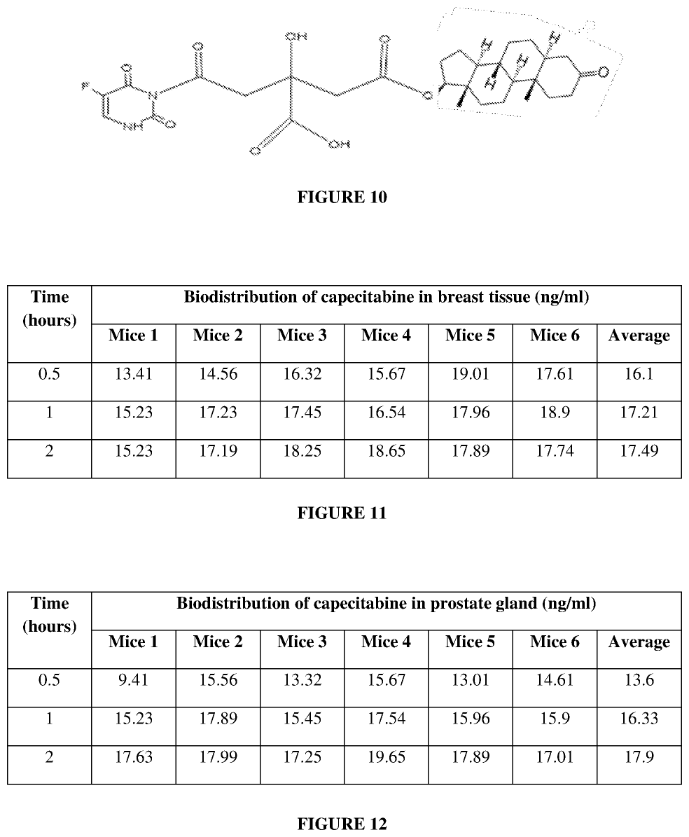

FIG. 10 illustrates the structure of the molecule of formula (I) comprising 5-fluorouracil-citric acid-dihydrotestosterone according to another embodiment of the invention. The molecule comprises citric acid as first chemical moiety, hormone derivative dihydrotestosterone as second chemical moiety and the anticancer agent 5-fluorourcail as third chemical moiety. 5-fluorouracil-citric acid-dihydrotestosterone is synthesized by esterification method using DMF as a solvent. The esterification reaction is achieved by blocking the amine group using EDCI. In addition, EDCI functions as a cross-linking agent. The chemical moieties are covalently linked to one another. The molecular weight of 5-fluorouracil-citric acid-dihydrotestosterone is 612.64 g/mol.

The hormone derivative estradiol is incorporated into molecule of formula (I) in treating breast cancer in females. The hormone derivative estradiol is a weak female hormone. Estradiol is a carrier ligand in active targeting. The nucleolus of estradiol interacts with the estrogen receptor of the breast tissue. The anticancer agents are released by the process of de-esterification. The anticancer agent acts as antimetabolites for the cancerous cells. The molecule of formula (I) exhibits synergistic effect in anticancer activity in combination with the hormone derivative.

The hormone derivative dihydrotestosterone is incorporated into molecule of formula (I) in treating prostate cancer in males. The hormone derivative dihydrotestosterone is male hormone and a carrier ligand in active targeting. The nucleolus of dihydrotestosterone interacts with the testosterone receptor of the prostate gland. The anticancer agents are released by the process of hydrolysis of esters. The anticancer agent acts as antimetabolites for the cancerous cells. The molecule of formula (I) exhibits synergistic activity of anticancer agent in combination with the hormone derivative.

In some embodiments, the molecule of formula (I) comprises the acid such as citric acid or tartaric acid. The functional group of the acid is connected to the second chemical moiety i.e., the hormone derivative.

In some embodiments, the hormone derivative is selected based on the type of cancer to be treated. The molecule of formula (I) of the present invention comprises female hormone derivative for treatment of breast cancer and male hormone derivative for the treatment of prostate cancer. The female hormone derivative used in the present invention is estradiol and the male hormone derivative is dihydroxy testosterone.

In some embodiments, the anticancer drug used is selected based on the type of cancer to be treated. The molecule of formula (I) of the present invention comprises the anticancer agent selected from a group but not limited to 5-flurouracil, capecitabine, gemcitabine, 6-mercaptopurine or calibridin for treatment of breast cancer. Similarly, the anticancer drug used is 5-flurouracil for treatment of prostate cancer.

The following examples are offered to illustrate various aspects of the invention. However, the examples are not intended to limit or define the scope of the invention in any manner.

### Example 1: A Molecule for the Targeted Therapy of Breast Cancer

The molecule of formula (I) is derived using the molecular modeling. The molecule of formula (I) is derived for the treatment of breast cancer and comprises citric acid as first chemical moiety, estradiol as female hormone derivative and 5-fluorouracil as the third chemical moiety. The chemical moieties are linked through covalent bond through the functional groups and the molecule of formula (I). The molecule of formula (I) thus synthesized targets the hormone receptors thus resulting in improved efficacy and is more specific to cancer cells with less harm to the normal cells.

### Example 2: A molecule for the targeted therapy of prostate cancer

The molecule of formula (I) is derived using the molecular modeling. The molecule of formula (I) is derived for the treatment of prostate cancer and comprises citric acid as first chemical moiety, dihydroxy testosterone as male hormone derivative and 5-fluorouracil as the third chemical moiety. The chemical moieties are linked through covalent bond through the functional groups and the molecule of formula (I). The molecule of formula (I) thus synthesized targets the hormone receptors thus resulting in improved efficacy and is more specific to cancer cells with less harm to the normal cells.

The biodistribution analysis is carried out for capecitabine to identify the target organs and estimate the safety and efficacy of capecitabine. The route of administration incorporated for ester of citric acid with capecitabine and estrogen and ester of citric acid with capecitabine and dihydrotestosterone is through intravenous (IV) bolus.

### Example 3: Biodistribution Analysis of Capecitabine

The biodistribution analysis was carried out for capecitabine in plasma. Biodistribution analysis is crucial for the identification of target organs and to determine safety and efficacy of the molecule. The biodistribution analysis was carried out in breast tissue for females and in prostate gland for males along with several other organs including blood, liver, lungs and spleen.

The biodistribution analysis was carried out in three groups of albino mice. The female albino mice of age 7-8 weeks and weight 19g-35g were analyzed for the distribution of capecitabine in breast tissue. The male albino mice of age 7-8 weeks and weight 20g-30g were analyzed for the distribution of capecitabine in prostate gland. The study groups comprised of 3 groups including group 1, group 2 and group 3. The study group 1 comprised 6 mice receiving normal control, study group 2 comprised 6 mice receiving pure drug solution and study group 3 comprised 6 mice receiving molecule of formula (I) anticancer drug. The female group of mice were administered with 229.91 mg of citric acid-capecitabine-estradiol for breast cancer and the male group of mice were administered with 227 mg of citric acid-capecitabine-dihydrotestosterone for prostate cancer.

The analysis of capecitabine in plasma was initiated by liquid-liquid extraction using ethyl acetate. The extraction was carried out on ethylene bridged hybrid (BEH) C18 (100×2.1 mm, 1.7 μm) column. An isocratic flow rate of 0.5 ml/min of acetonitrile (0.1%) and 0.002M ammonium acetate as mobile phase was incorporated. Mass spectrometry analysis was carried out in multiple reaction monitoring modes. The precursor to product ion transitions with mass to ion (m/z) ratio of 360.04 to 244.08 was found for capecitabine. The linearity regression was found to be greater than 0.99 (r²>0.99). The biodistribution data for capecitabine was obtained for concentrations ranging from 5 ng/ml and 40 ng/ml.

FIG. 11 tabulates the biodistribution of capecitabine in breast tissue of mice. The biodistribution of capecitabine was analyzed at time intervals of 0.5 hours, 1 hour and 2 hours. The biodistribution data was recorded for 6 mice. The female group of mice were administered with 229.91 mg of citric acid-capecitabine-estradiol for breast cancer and the male group of mice were administered with 227 mg of citric acid-capecitabine-dihydrotestosterone for prostate cancer. At time 0.5 hours, the average biodistribution of capecitabine was found to be 16.1 ng/ml. At time 1 hour, the average biodistribution of capecitabine was found to be 17.21 ng/ml. At time 2 hours, the average biodistribution of capecitabine was found to be 17.49 ng/ml. The average biodistribution of capecitabine indicates the distribution of capecitabine at the target organ, breast tissue has been achieved at various time intervals.

FIG. 12 tabulates the biodistribution of capecitabine in prostate gland of mice. The biodistribution of capecitabine was analyzed at time intervals of 0.5 hours, 1 hour and 2 hours. The biodistribution data was recorded for 6 mice. The female group of mice were administered with 229.91 mg of citric acid-capecitabine-estradiol for breast cancer and the male group of mice were administered with 227 mg of citric acid-capecitabine-dihydrotestosterone for prostate cancer. At time 0.5 hours, the average biodistribution of capecitabine was found to be 13.6 ng/ml. At time 1 hour, the average biodistribution of capecitabine was found to be 16.33 ng/ml. At time 2 hours, the average biodistribution of capecitabine was found to be 17.9 ng/ml. The average biodistribution of capecitabine indicates the distribution of capecitabine at the target organ, prostate gland has been achieved at various time intervals.

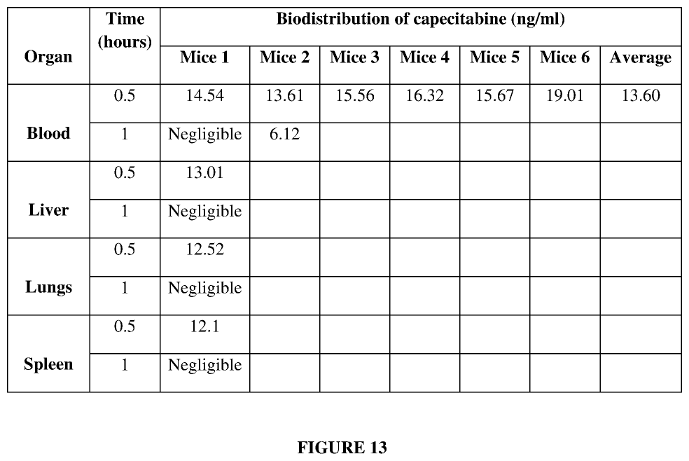

The biodistribution of capecitabine was also analyzed for several other organs such as blood, liver, lungs and the spleen. FIG. 13 tabulates biodistribution of capecitabine in several organs of mice. The biodistribution of capecitabine was analyzed at time intervals of 0.5 hours, 1 hour and 2 hours. The biodistribution data was recorded for 6 mice. The female group of mice were administered with 229.91 mg of citric acid-capecitabine-estradiol for breast cancer and the male group of mice were administered with 227 mg of citric acid-capecitabine-dihydrotestosterone for prostate cancer. At time 0.5 hours, the average biodistribution of capecitabine in blood was found to be 13.60 ng/ml. During the later hours the biodistribution of capecitabine was found to be in negligible concentrations. At time 0.5 hours, the biodistribution of capecitabine in liver in mice 1 was found to be 13.01 ng/ml followed by negligible concentrations at the later hours. At time 0.5 hours, the biodistribution of capecitabine in lungs in mice 1 was found to be 12.52 ng/ml followed by negligible concentrations at the later hours. At time 0.5 hours, the biodistribution of capecitabine in spleen in mice 1 was found to be 12.1 ng/ml followed by negligible concentrations at the later hours. The biodistribution data of capecitabine in organs including blood, liver, lungs and spleen indicate that the anticancer agent capecitabine has been found in maximum concentrations in the target tissue and not distributed among other organs.

The molecule of formula (I) is synthesized using multiple combinations of drugs but not restricted to said examples as per the present invention. As the molecule of formula (I) is synthesized with target ligand receptors, the molecule specifically targets the hormone receptors. As a result of which, the molecule of formula (I) induces cytotoxic effect in only cancer cells without affecting the healthy or normal cells in the nearby environment.

Further, the molecule of formula (I) synthesized exhibits a low molecular weight and due to which the molecule of formula (I) easily passes through the cell membrane and binds to the hormone receptors. In addition, the conjugation of the ligand aids in specific targeting of the hormone receptors in achieving the targeted therapy. The molecule of formula (I) is chemically stable, cost effective, exhibits improved efficacy due to specific target, extended duration of therapeutic activity without inducing any immune reaction.

We claim:

1. A molecule of formula (I) for the targeted treatment of cancer, the molecule of formula (I) comprises:
a) an acid;
b) a hormone derivative; and
c) an anticancer agent
wherein the molecule of formula (I) is of low molecular weight, highly stable, exhibits extended therapeutic activity.

2. The molecule of formula (I) as claimed in claim 1, wherein said cancer is breast cancer or prostate cancer.

3. The molecule of formula (I) as claimed in claim 1, wherein said acid is selected from a group comprising citric acid and tartaric acid.

4. The molecule of formula (I) as claimed in claim 1, wherein said hormone derivative is estradiol for targeting the ligand receptor in breast cancer and dihydrotestosterone for targeting the ligand receptor in prostate cancer.

5. The molecule of formula (I) as claimed in claim 1, wherein the said anticancer agent is selected from a group comprising 5-flurouracil, capecitabine, cladibrine, ethionine and mercaptopurine.

6. The molecule of formula (I) as claimed in claim 1, wherein said molecule of formula (I) is synthesized by the esterification of the acid, the hormone derivative and the anticancer agent.

7. The molecule of formula (I) as claimed in claim 1, wherein said molecule of formula (I) exhibits synergistic effect of the anticancer agent and the hormone derivative.
